(19) United States
(12) Patent Application Publication
(10) Pub. No.: US 2010/0310527 A1
(43) Pub. Date: Dec. 9, 2010

(54) LIPOSOME MEDIATED DELIVERY OF LINEAGE DETERMINING FACTORS

(75) Inventors: Eckhard U. Alt, Houston, TX (US); Kirstin Schimrosczyk, Madison, WI (US); Michael E. Coleman, The Woodlands, TX (US)

Correspondence Address: FISH & RICHARDSON P.C. (NY), P.O. BOX 1022, MINNEAPOLIS, MN, 55440-1022, US

(73) Assignees: BOARD OF REGENTS OF THE UNIVERSITY OF TEXAS SYSTEM, Austin, TX (US); INGENERON, INC., Houston, TX (US)

(21) Appl. No.: 12/684,263

(22) Filed: Jan. 8, 2010

Related U.S. Application Data
(60) Provisional application No. 61/143,591, filed on Jan. 9, 2009.

(51) Int. Cl. A61K 35/12; C12N 5/071; A61P 9/00

(52) U.S. Cl. 424/93.7; 435/325; 435/395

(57) ABSTRACT

Methods and compositions are provided for lineage predetermination of cellular transplants including through liposome mediated transfection with aqueous protein extracts from populations of differentiated mammalian cells, or cellular fractions thereof, wherein the differentiated mammalian cells are enriched in one or more of adipocytes, chondrocytes, endothelial cells, hepatocytes, cardiomyocytes, smooth muscle cells, skeletal muscle cells, cardiac pacemaker cells, Schwann cells, pancreatic islet cells, hematopoietic cells, myeloblasts, neurons, and osteoblasts.

### CROSS REFERENCE TO RELATED APPLICATIONS

This application claims priority based on U.S. Provisional Application Ser. No. 61/143,591 filed Jan. 9, 2009, which is incorporated herein by reference.

### FIELD OF THE INVENTION

This invention relates compositions and methods for modulating the differentiation of isolated reparative cell populations, including stem cells.

### BACKGROUND OF THE INVENTION

Without limiting the scope of the invention, its background is described in connection with novel compositions and methods for selectively directing the differentiation of isolated cells prior to implantation.

The field of regenerative medicine has been extensively studying the potential of cell therapy for repair of injured or diseased tissue. The use of stem cells in such indications has been emphasized. Mesenchymal stem or stromal cells (MSC), originally isolated from bone marrow, are considered to be pluripotent and are thus potentially able to differentiate into a myriad of cell types including osteoblasts, chondrocytes, myocytes, adipocytes, and islet cells. More recently it has been found that MSC can also be isolated from the stroma of adipose tissue, which is considerably more readily obtained than is bone marrow. Indeed, by virtue of its relatively high content of MSCs, adipose tissue has been shown to be a convenient source of cells that have shown utility for cell therapy, at least in a research setting. Like MSC from bone marrow, adipose derived MSC, or “ADSC” are pluripotent. ADSC have recently yielded cell preparations useful for the repair of articular cartilage. Additionally, these stromal cells have been cultured in inductive media to differentiate into cells having neuronal characteristics. Finally, ADSC have been induced to differentiate into hematopoietic cells, osteogenic cells, endothelial cells, adipocytes and myocytes of skeletal and smooth muscle. These in vitro efforts may recapitulate natural regenerative processes whereby adult stem cells effect regeneration of damaged tissue by differentiating into needed cell types.

Because stem cells are relatively rare, the ability to generate a particular desired differentiated cell type from progenitor cells has been a goal of regenerative medicine. Heretofore, prolonged culture in various lineage-specific inductive media has been utilized in the generation of enriched populations of differentiated cells. However, the repertoire of inductive media is limited and the requirement for prolonged culture limits the usefulness of the procedure.

Efforts to enhance the differentiation rate have been undertaken. For example, a cardiomyogenic cell line (CMG) from murine bone marrow stromal cells was induced to develop cardiomyocyte-like structures and phenotypes similar to those of fetal ventricular cardiomyocytes by treatment with 5-azacytidine. (Makino S, et al. “Cardiomyocytes can be generated from marrow stromal cells in vitro.” J Clin Invest 103 (1999) 697-705). However, the requirement of treatment with a cytotoxic and potentially mutagenic agent such as 5-azacytidine limits the desirability of the procedure.

Cell differentiation has been shown to be influenced by cell contact. Indeed, it has long been recognized that cell phenotype can be altered by the immediate microenvironment. Complex interactions between cells and nuclear signaling elements play a crucial role in determining cell fate. For example, it has been demonstrated that bone marrow stromal cells are able to transdifferentiate into myocytes that express cardiac transcription factors by direct cellular contact with cardiomyocytes. See e.g. Xu M. et al. “Differentiation of Bone Marrow Stromal Cells Into the Cardiac Phenotype Requires Intercellular Communication With Myocytes” Circulation 110 (2004) 2658-2665. Embryonic or neonatal umbilical vein endothelial, but not adult endothelial cells have been shown to transdifferentiate into beating cardiomyocytes when co-cultured with neonatal rat cardiomyocytes. See Condorelli, G. et al. “Cardiomyocytes induce endothelial cells to transdifferentiate into cardiac muscle: implications for myocardium regeneration” Proc. Natl. Acad. Sci. USA 98 (2001) 10733-10738.

The potential to reprogram somatic cells using cell extracts has been suggested on the basis that exposure of permeabilized fibroblasts to embryonic stem cell extracts resulted in transient morphologic changes and expression of stem cell markers. (Collas, US Patent Application Publication US2002/0142397 entitled “Methods of Altering Cell Fate”). According to the Collas disclosure, the reprogramming extract is introduced into the recipient cell by permeabilization of the recipient cell membrane with a detergent, such as digitonin, or a bacterial toxin, such as Streptolysin O (SLO), which is a cholesterol-binding toxin that forms large pores in the plasma membrane of mammalian cells. Collas' laboratory has also reported that human adipose tissue stem cells can adopt cardiomyocyte properties following exposure to an adult rat cardiomyocyte protein extract via streptolysin permeabilization. See Gaustad et al. “Differentiation of human adipose tissue stem cells using extracts of rat cardiomyocytes” Biochem Biophys Res Commun. 314(2) (2004) 420-7. However, the permeabilization procedure has a very significant impact on viability. In a detailed procedure paper Collas has reported that approximately 50% of recipient cells are lysed by SLO treatment. See Hakelien et al. “Modulation of Cell Fate Using Nuclear and Cytoplasmic Extracts” Methods in Molecular Biology 325 (2006) 99-114.

The ability to generate precommitted cells for treatment of cardiovascular disease is particularly needed on the basis that cardiovascular disease is the leading cause of mortality in the Western world. An estimated 17.5 million people died from cardiovascular disease in 2005. Of these deaths, about 7.6 million were due to coronary heart disease (WHO data). Although pharmacological and lifestyle interventions are of proven benefit, in most cases the progression of disease is only slowed toward an endpoint of the potentially lethal cardiac functional impairment of congestive heart failure. The only available cure for this disease is heart transplantation, which is an obviously limited solution. An efficacious cell-based therapy for heart failure is therefore an attractive prospect. Animal experiments have shown that implanting various undifferentiated stem cells into injured tissue is followed by two primary events. First, angiogenesis is induced with a resultant increase in the survival of resident cells. Second, functional parameters such as elasticity of damaged myocardium are improved, therefore preventing progressive dilatation and thinning (Al Radi O O, et al. “Cardiac cell transplantation: closer to bedside” Ann Thorac Surg. 75(2) (2003) S674-S677.1). The direct involvement of engrafted stem cells in subsequent in situ differentiation into cellular components of regenerated tissue is thought to play a role. However, promising these studies may be, the ability to impart lineage determination to stem cells prior to implantation is expected to increase the numbers of useful cells, increase survival of such cells after implantation, and shorten the time until appropriate function is attained.

While existing methods have allowed considerable study of transdifferentiation in research environments, methods and apparatus for preconditioning of isolated stem cells in sufficient quantity and quality for clinical use continues to represent an unmet need. What is needed are improved methods for inducing transdifferentiation such that sufficient numbers of autologous regenerative cells can be obtained for use in human and animal medicine.

### BRIEF SUMMARY OF THE INVENTION

The present invention is directed to methods and compositions for generating isolated populations of lineage pre-determined cells for transplantation. In one embodiment of the invention a recipient cell population is isolated that is enriched in cells that are characterized by phenotypic plasticity, including stem cells and cells that are able to transdifferentiate. In one embodiment of the invention, the recipient cell population is enriched in bone marrow or adipose derived mesenchymal stem cells. The recipient cells are transfected with a liposome composition comprising a lineage predetermining protein extract obtained from differentiated donor cells thereby obtaining a population of transfected recipient cells that express at least one lineage predetermined marker derived from the differentiated donor cells. The novel liposomal transfection method provides high transfection rates as well as low toxicity thus providing larger more vigorous cells for transplantation.

In one embodiment of the invention, the lineage predetermining cell extract is a cytoplasmic extract obtained from the differentiated donor cells. In other embodiments, the lineage predetermining extract is a nuclear extract. Surprisingly, the lineage predetermining extract is effective whether obtained from xenogeneic or allogenic, neonatal or adult differentiated donor cells. The lineage predetermining protein extract may be obtained cell populations enriched in one or more of adipocytes, chondrocytes, endothelial cells, hepatocytes, cardiomyocytes, smooth muscle cells, skeletal muscle cells, cardiac pacemaker cells, Schwann cells, pancreatic islet cells, hematopoietic cells, myeloblasts, neurons, and osteoblasts. The cell populations may be enriched on the basis of being derived from a particular specialized organ. For example a cell population enriched in cardiomyocytes may be obtained from whole heart. Alternatively, particular cell populations may be isolated from the organ to provide further enrichment of specialized cell types.

In one embodiment of the invention lineage predetermined reparative or isolated stem cell preparations are utilized for cell therapy without prior expansion in cell culture. In other embodiments, the lineage predetermined cells are further expanded in culture prior to implantation. This may be particularly indicated where adipose derived stem cells are desired and the donor has very little adipose tissue. In one embodiment of the invention, the transfected cells are further encouraged down a particular differentiation pathway by culturing the population of transfected recipient cells in a differentiation induction media for some time prior to implantation. In other embodiments, the transfected cells are subject to one or more further transfections to stabilize or prolong their lineage commitment.

In one embodiment, a method of treating a diseased tissue in a patient with a lineage pre-determined cell transplant is provided including by removing a sample of adipose or bone marrow tissue from the patient and isolating from the tissue a recipient cell population that is enriched in cells characterized by phenotypic plasticity. In one embodiment, the recipient cell population is an isolated stem cell population while in other embodiments the recipient cell population is a freshly prepared adipose stromal cell fraction. The recipient cell population is treated with a liposome composition comprising a lineage predetermining protein cell extract, whereby a significant proportion of the cell population is induced to express at least one lineage predetermined marker as a consequence of the treatment. The transfected recipient cells are implanted into the diseased tissue of the patient. In one particular embodiment, the diseased tissue is a tissue damaged by myocardial infarction.

In one embodiment of the invention, methods are provided for preparing lineage predetermining compositions including by preparing an aqueous protein extract from a population of differentiated mammalian cells, or cellular fractions thereof, wherein the differentiated mammalian cells are enriched in one or more of adipocytes, chondrocytes, endothelial cells, hepatocytes, cardiomyocytes, smooth muscle cells, skeletal muscle cells, cardiac pacemaker cells, Schwann cells, pancreatic islet cells, hematopoietic cells, myeloblasts, neurons, and osteoblasts. The aqueous protein extract is added to a dry lipid film and mixed until a liposomal solution is formed which is then characterized, standardized and stabilized for use as a reagent.

A biocompatible or biodegradable cell or tissue scaffold is also provided that is combined with a liposome composition that includes at least one lineage predetermining protein cell extract. Biocompatible materials include but are not limited polytetrafluoroethylene, woven polyester, spun silicone, open foam silicone encased in microporous expanded PTFE, stainless steel, polypropylene, polyurethane, polycarbonate, nickel titanium shape memory alloys and cobalt-chromium-nickel alloys, and combinations thereof. Biodegradable materials include but are not limited to silk fibroin-chitosan, acellular dermal matrices, collagen, polyglactin, and hyaluronic acid. Where the liposomal solution containing a lineage predetermining protein extract is combined with a biocompatible or biodegradable cell or tissue scaffold, the combination of scaffold and liposome solution may be made fresh or may be prepared in advance and stored as a sterile ready to use commodity. When stromal vascular cells are applied to the scaffold, either in vitro or in vivo, the cells come in contact with the liposomal solution and are transfected thus driving their differentiation according to the lineage predetermining protein extract or extracts incorporated into the liposomal solution.

### BRIEF DESCRIPTION THE DRAWINGS

For a more complete understanding of the present invention, including features and advantages, reference is now made to the detailed description of the invention along with the accompanying figures:

FIG. 1 depicts an embodiment of a test system adapted to determination of cardiomyocyte lineage predetermination.

FIG. 2, A-C, presents FACS analysis of ADSCs transfected with 3.5 μg protein (A) Cells maintained normal cell shape but (B, C) expressed a bright fluorescence—84% of cells (P3) were significantly brighter than control ADSCs.

FIG. 3 depicts immunofluorescence results with ADSC transfected with whole-heart nuclear and cytoplasmic extracts.

FIG. 4 depicts immunofluorescence results with ADSC transfected with isolated cardiomyocyte nuclear and cytoplasmic extracts.

FIG. 5 depicts a comparison of immunofluorescence results with ADSC transfected cardiomyocyte extracts compared to whole heart extracts

FIG. 6, A-F, depicts expression of various differentiation marker mRNA by RT-PCR.

### DETAILED DESCRIPTION OF THE INVENTION

While the making and using of various embodiments of the present invention are discussed in detail below, it should be appreciated that the present invention provides many applicable inventive concepts which can be employed in a wide variety of specific contexts. The specific embodiment discussed herein are merely illustrative of specific ways to make and use the invention and do not delimit the scope of the invention.

Increasing evidence suggests that stem cells are residents of a micro-vascular niche, on stand-by for tissue repair as needed. However, with extensive tissue damage, the local pool of stem cells available for repair is considered insufficient to fully correct the deficiency. In the present invention, liposome mediated lineage predetermination of isolated cell populations having lineage plasticity is adapted to provide early lineage commitment of the lineage predetermined cells and provide rapid functional attainment in engrafted tissue.

Sources of Stem Cells and Cells having Development Plasticity: It is now understood that stem cells are present in many tissues and play an important role in maintaining local homeostasis. Adult tissue-derived stem cells were previously regarded to be restricted to their lineage in their differentiation potency. It is now believed that tissue-derived stem cells have a phenotypic potential that extends beyond the differentiated somatic cell phenotypes of their resident tissue. For practical reasons the stem cells most suitable on the basis of sufficient numbers for use in ex vivo lineage predetermination include bone marrow-derived cells and adipose-derived cells.

In 2005, the International Society for Cellular Therapy (ISCT) stated that the currently recommended term for plastic-adherent cells isolated from bone marrow and other tissues is multipotent mesenchymal stromal cells (MSC) in lieu of the prior “stem cell” term. MSC have been traditionally defined as spindle-shaped or fibroblast-like plastic adherent cells. Although originally isolated from bone marrow, MSC have now been isolated from a variety of tissues including bone periosteum, trabecular bone, adipose tissue, synovium, skeletal muscle, dental pulp and cord blood.

As used herein the term Mesenchymal Stromal Cell (MSC) means the definition adopted by the International Society for Cellular Therapy and published in a position paper by Dominici et al, Cytotherapy 8 (2006) 315. In accordance with the position paper, MSC must exhibit:
1) adherence to plastic in standard culture conditions using tissue culture flasks;
2) a specific surface antigen (Ag) phenotype as follows:
   positive (≧95%+) for CD 105 (endoglin, formerly identified by MoAb SH2), CD73 (ecto 5′ nucleotidase, formerly identified by binding of MoAbs SH3 and SH4), CD90 (Thy-1), and
   negative (≦2%+) for CD14 or CH11b (monocyte and macrophage marker), CD34 (primitive hematopoietic progenitor and endothelial cell marker), CD45 (pan-leukocyte marker), CD79α or CD19 (B cells), and HLA-DR (unless stimulated with IFN-γ); and
3) tri-lineage mesenchymal differentiation capacity: ability to differentiate in vitro into osteoblasts, adipocytes and chondrocytes in inductive media.

Bone marrow-derived mesenchymal stem cells (MSCs) are the non-hematopoietic population derived from the bone marrow stroma. Caplan and Haynesworth (Osiris U.S. Pat. No. 5,486,359) described isolation of pluripotent mesenchymal stem cells from bone marrow using Percoll gradient separation and plating of the lowest density fraction on plastic. The isolated mesenchymal stem cells were plastic adherent and had fibroblast-like morphology. A panel of monoclonal antibodies was developed to these cells and including antibodies termed SH2, SH3 and SH4. These antibodies now have the following correlated CD markers: SH2 (CD105), SH3 and SH4 (CD73). Davis-Sproul et al (Osiris U.S. Pat. No. 6,387,367) described isolation of pluripotent mesenchymal stem cells from bone marrow or blood using density gradient separation and collection of the light density cells followed by immunomagnetic bead separation of CD45+ cells. These cells were also positive for SH3 (a.k.a. CD 73) or SH2 (a.k.a. CD 105) and could be pre-selected for these markers. Bone marrow-derived MSC are of interest in terms of revascularization and cardiac repair because such MSCs have the potential to release cytokines and growth factors stimulating endogenous repair mechanisms and have a relatively low immunogenicity that extends their utility to allogenic applications. Limiting the use of bone marrow-derived MSC in clinical therapeutic applications is the painful and potentially dangerous extraction procedure.

One particularly suitable source of stem cells for purposes of the present invention is adipose tissue, including that obtained by liposuction procedures. Such adipose tissue contains a significant number of mesenchymal stromal/stem cells accessed via a relatively low-risk surgical intervention. Adipose tissue is highly vascularized and is thus a source of endothelial cells, smooth muscle cells, its progenitors and of early multipotent mesenchymal stem cells. Adipose tissue may provide as many as 300,000 reparative cells per gram, which have an appropriate cell type composition.

Adipose tissue is characterized by the presence of mature adipocytes bound in a connective tissue framework termed the “stroma.” In the present invention, stromal cells generally refer cells resident in the connective tissue of an organ or tissue. Non-limiting examples of such cells include fibroblasts, macrophages, monocytes, pericytes, endothelial cells, inflammatory cells, progenitors and early undifferentiated mesenchymal stem cells. Such cells also participate in tissue maintenance and repair, typically as supportive cells. The stroma of adipose tissue includes an array of cells that do not include the lipid inclusions that characterize adipocytes. These include preadiopcytes, fibroblasts, vascular smooth muscle cells, endothelial cells, monocyte/macrophages and lymphocytes.

As used herein, “reparative cell population” refers to a mixture of cells that includes “tissue engrafting cells” that are herein defined to include MSC as well as cells such as fibroblasts and endothelial cells that are able to proliferate and engraft a target tissue when returned to the body. The reparative cell population may also include one or more “supportive cell” populations. Supportive cells are herein defined as cells that do not permanently engraft in the target tissue but may aid in the tissue remodeling process that is essential to healing of damaged tissue. These may include, for example, lymphocytes and macrophages. As used herein the term “reparative cell population” is not limited to plastic adherent cells and may be the same as adipose stromal vascular fraction cells under some circumstances.

In the present invention, “progenitor cells” generally refer to uncommitted mesenchymal stem cells in various mesenchymal tissues, such as muscle, bone, cartilage and adipose tissue and vascular progenitor cells that can be differentiated into vascular cell types. Such cells are generally believed to constitute a cellular reserve fraction and function as target engrafting cells.

The following examples are included for the sake of completeness of disclosure and to illustrate the methods of making the compositions and composites of the present invention as well as to present certain characteristics of the compositions. In no way are these examples intended to limit the scope or teaching of this disclosure.

### Example 1

### Establishment of a System for Lineage Predetermination

Lineage determination of constituent cells is required for the cells to function as part of a specialized tissue. Two different processes, differentiation and transdifferentiation, participate in cellular differentiation and development. In the adult, differentiation involves the lineage differentiation of pluripotent stem cells into various committed lineages. Transdifferentiation describes the conversion from one differentiated cell type to another functional cell type, perhaps through an intermediary transient dedifferentiation to a more primitive phenotype. Though transdifferentiation strictly relates to a dynamic bi-directional developmental capacity, known as plasticity, it is widely used in a less-strict sense in reference to the differentiation of stem cells in general. Differentiation and transdifferentiation are pathways that occur spontaneously or can be induced by certain factors. It has been shown that various substances are effective triggers for cardiomyogenic differentiation in stem cells and that exposure to different stimuli in the microenvironment can lead to reprogramming of a cell. As used herein, the terms “differentiation” and “transdifferentiation” are used interchangeably to refer to the induced change in phenotype of adult cells that have at least some phenotypic plasticity. Typically, the methods of the present invention will be directed to isolated cell populations that are substantially enriched in mesenchymal stem or stromal cells that are herein shown to undergo phenotypic conversion by liposome mediated transfection with cytoplasmic and/or nuclear extracts derived from differentiated cells.

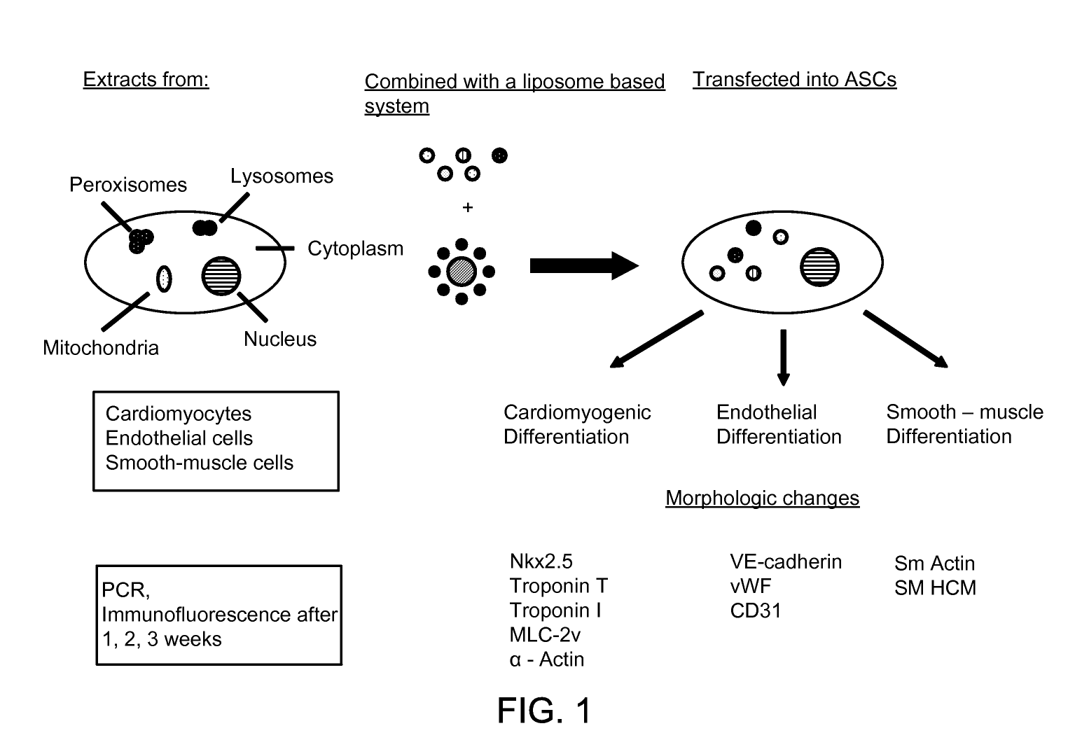

The system disclosed herein was established to improve the efficiency of lineage predetermination of MSC. A test system, adapted to assessing cardiomyocyte lineage predetermination, is depicted in FIG. 1. For assessment of different lineages, such as for example, hepatic or neurogenic, different panels of markers would be selected.

It was established by the methods disclosed herein that liposome mediated transfer of cytoplasmic and nuclear factors from differentiated cells provides an efficient method of ex-vivo lineage pre-determination of ADSC prior to implantation. In particular, the ability of adipose-derived stem cells to differentiate into cardiomyocyte-like cells and other cardiac cell lineages such as vascular smooth-muscle cells and endothelial cells is shown. As examples of the utility of the method in inducing phenotypic conversion of cells having differentiation plasticity into various lineages, differentiation was induced in ADSC with protein extracted from neonatal rat hearts, human smooth-muscle, and human endothelial cells. The liposome-based transfection system disclosed herein was established as an efficient means to induce differentiation in ADSCs. The achieved high transfection efficiencies of approximately 90% in addition to very low cytotoxicities are important factors in a clinically viable system. These advantages contrast with prior methods.

For example, differentiation-inductive protein transfers by means of electroporation or streptolysin permeabilization have been reported previously. (Gaustad K G, et al. “Differentiation of human adipose tissue stem cells using extracts of rat cardiomyocytes” Biochem Biophys Res Commun. 314(2) (2004) 420-427). The differentiation rate observed using these methods was low and considerably lower than in the present study using a liposome-based cellular transfection approach. For the first time, a liposome-based carrier system is described to induce the differentiation of adipose-derived stem cells through the transfer of proteins while avoiding major damage to the cell.

In defining the system, proteins used for transfection were extracted separately from different cell components. Transfection was performed with separated cell compartment extracts, leading to the finding that while cell free extracts from both the cytoplasm and nucleus have a high differentiation potential, nuclear extracts are particularly useful. The greater inductive potency of nuclear protein extracts versus cytoplasmic extracts observed with troponin I expression levels is generally representative of the overall pattern seen across the variety of cardiovascular markers used as differentiation markers. Nuclear protein extracts were consistently more potently inductive of cardiovascular differentiation than cytoplasmic protein preparations. Additionally, it was demonstrated that neither neonatal nor allogenic species-specific proteins are necessarily needed for induction of differentiation as both xenogenic neonatal rat cardiac and human adult endothelial and smooth-muscle cell-derived protein preparations were successfully used for induction. The feasibility of trans-species induction was shown by utilizing protein extracts from neonatal rat cardiomyocytes and whole-heart preparations for differentiation to a cardiogenic lineage. Thus, in one embodiment of the invention, allogenic or xenogenic lineage specific extracts are prepared and characterized and made available as a standardized reagent for use in liposome mediated lineage pre-determination of isolated autologous ADSC.

As further established herein, the alterations in phenotype induced by liposome mediated transfection are transient. On one level this demonstrated the safety of the procedure as no transformation of a type that might lead to malignant transformation was observed. On the other hand, the transient phenotypic conversion establishes a system for assessing the window within which the transfected cells must be transplanted after a single transfection in order to realize the benefits of lineage pre-determination. Once the lineage predetermined cells are implanted, it is expected that the specialized microenvironment of the target tissue will support maintenance of the pre-determined phenotype.

It has long been recognized that the immediate microenvironment can radically alter cell phenotype. Evidence suggests that three major factors contribute to the microenvironment and thus to the differentiation of stem cells: mechanical and microanatomic structural features of the niche, intra- and peri-niche paracrine-acting humoral factors, and the direct exchange of genetic information between stem cells and other resident cells of the immediate environment. Reports from the literature highlight this phenomenon whereby undifferentiated human mesenchymal and bone marrow-derived stem cells placed into the murine cutaneous wound environment are induced to differentiate along both mesodermal and ectodermal germ layer derivatives via cues of the microenvironment. (Altman A M, Matthias N, Yan Y, Song Y, Bai X, Chiu E S, Slakey D P, Alt E U. “Dermal matrix as a carrier for in vivo delivery of human adipose-derived stem cells” Biomaterials 29(10) (2008) 1431-1442; Li H, et al. “Adult bone-marrow-derived mesenchymal stem cells contribute to wound healing of skin appendages” Cell Tissue Res. 326(3) (2006) 725-736). The methods of the present invention are designed to jump-start this process and thereby provide a larger number of transplanted cells that are directed to the desired phenotypic lineage.

Furthermore, repair of certain types of tissue injury is particularly challenging. Though stem cells have been proven to have a differentiation-inducing potential, such potential is influenced by environmental limitations. In vivo studies investigating the potential of stem cells to regenerate infarcted myocardium have reported engraftment and differentiation of stem cells in infarcted hearts. However, the differentiation rate was poor and located only in the border zone, but not in the center of the infarct. (Wollert K C, Drexler H. “Clinical applications of stem cells for the heart” Circ Res. 96(2) (2005) 151-163).

Repair of scar tissue at the infarct center region remains the most challenging because transfected cells must face limited blood supply and a lack of environmental signals critical for differentiation into vascular cells or cardiomyocytes. In addition, the center zones of the infarct are inherently deficient in viable cardiomyocytes, smooth-muscle cells and endothelial cells that could induce injected stem cells to differentiate and regenerate the myocardium. Viable cardiomyocytes with the potential to transfer inductive signaling factors to stem cells are limited in the setting of myocardial infarction. In this depleted milieu, the lineage pre-differentiated stem cells provided by the present invention are designed to improve the regenerative outcome. Prior stimulation of differentiation would diminish the need for post-engraftment induction through cellular contact. Consequently the regeneration of damaged myocardium is expected to be enhanced.

### Example 2

### Generation of Cardiomyocytes from Adipose Derived Stromal Cells

Isolation of ADSC: Subcutaneous adipose tissue was obtained from patients undergoing elective body-contouring and reconstructive procedures. Adipose tissue was minced and incubated for 90 min at 37° C. on a shaker in 20 ml phosphate-buffered saline (PBS) with 25 mg of Collagenase VIII (Sigma, St. Louis, Mo., USA) and 5 mM calcium chloride. The digested tissue was passed through a 100 mm filter (Millipore, Billerica, Mass., USA) and centrifuged at 450 g for 10 min. The supernatant containing adipocytes and debris was discarded and the pelleted cells were washed twice with 40 ml Hanks Balanced Salt Solution (Cellgro, Manassas, Va., USA) and finally resuspended in growth media. Growth media contained 500 ml alpha-modification of Eagle's medium (αMEM, Cellgro), 100 ml fetal bovine serum (Atlanta Biologicals), 5 ml L-glutamine (0.2 M), 5 ml penicillin (10,000 U/ml) with streptomycin (10 mg/ml). Plastic-adherent human adipose tissue-derived stem cells (ADSCs) were grown in Nunclon culture vials at 37° C. in a humidified atmosphere containing 5% CO₂ followed by daily washings to remove red blood cells and nonattached cells. ADSCs were then initially plated at a density of 1,000 cells/cm² and were passaged once a week. Cell viability was determined by fluorescence-assisted cell sorting (FACS) analysis after staining with 1 mM propridium iodide (PI).

FACS characterization of third passage hADSCs from three samples was performed as described in Bai et al. “Electrophysiological Properties of Human Adipose Tissue-Derived Stem Cells” Am. J. Cell Physiol 293 (2007) C-1539-C1550. The primary antibodies were fluorescein isothiocyanate (FITC) conjugated-anti-human CD44, CD34, CD90 (US Biological) or phycoerythrin (PE)-conjugated-anti-human CD11b, CD105 (eBioscience), CD14, and CD45 (US Biological). Isotype-matched normal mouse IgGs were used as controls (Chemicon). The isolated ADSCs are characteristically plastic-adherent, spindle-shaped cells. When cultured they were positive for CD44 (95.44±2.66%), CD90 (95.87±3.51%), CD105 (98.54±1.89%) and negative for CD11b (0.58±0.69%), CD14 (0.08±0.02%), CD34 (0.08±0.07%) and CD45 (0.11±0.10%).

To evaluate the multipotent capacity of the isolated hADSCs, cells were cultured in adipogenic (low glucose Dulbecco's modification of Eagle's medium (DMEM) supplemented with 10% FBS, 2 mM L-glutamine, 100 U/ml penicillin, 100 mg/ml streptomycin (Cellgro), 100 mM L-ascorbate acid (Sigma, St. Louis, Mo., USA), 1 mM dexamethasone, 0.5 mM 1-methyl-3-isobutylxanthine (IBMX), 100 mM indomethacin and 10 mg/ml human recombinant insulin (Sigma, St. Louis, Mo., USA) or osteogenic induction medium (high glucose DMEM plus 10% FBS supplemented with 0.1 mM dexamethasone, 200 mM L-ascorbic acid and 10 mM b-glycerol phosphate (Sigma)) for 3 weeks. hADSCs in adipogenic induction medium displayed characteristic multiple intracellular oil droplets showing red vesicles when stained with Oil Red O stain (Sigma). No lipid droplets were observed in cells cultured in control medium. Cells cultured in osteogenic induction medium showed the calcification deposits (stained with Alizarin red stain (Sigma) characteristic of differentiated osteoblasts, which was not observed in cells cultured in control medium.

Cells cultured in the hepatogenic induction medium (DMEM (1 g/L glucose), 1% FBS, 10 ng/ml bFGF, 20 ng/ml aFGF (Chemicon), 1% ITS, 10 ng/ml EGF (Sigma), 10 ng/ml OSM, 20 ng/ml HGF (R&D)) for 21 days became small and polygonal epitheloid cell-like. Immunofluorescence staining showed that albumin, a specific marker for hepatocytes, could be detected in cells cultured hepatogenic induction medium but not in control medium. Cells exposed to the neurogenic medium (DMEM/F12, 1% FBS, 2% B27 (Invitrogen), 10 ng/ml bFGF (Chemicon), 10 ng/ml EGF, 10 mM forskolin, 1 mM cAMP (for human 2 mM), 5 mg/ml insulin, 0.5 mM 1-methyl-3-isobutylxanthine, 1 mM 2-mercaptoethanol, 50 mM Vitamin C (Sigma), 10 ng/ml NGF (R&D)) for 21 days exhibited radical elongated processes which are positive for MAP-II, a specific neuron marker. Cells cultured in control medium did not express MAP-II.

Isolation of Lineage Determining Factors: Cardiomyocytes were isolated from rat hearts of 1 to 3-day-old rats by collagenase treatment using the neonatal cardiomyocyte isolation system (LK003300, Worthington, Lakewood, N.J., USA) according to the manufacturer's protocol. The isolated cardiomyocytes were cultured for 3 days in cardiomyocyte medium provided by the kit. Attached cells were beating.

For protein isolation and labeling, proteins were obtained either from isolated neonatal rat cardiomyocytes or whole neonatal rat hearts. Protein extraction was accomplished using NE-PER nuclear extraction reagents (Cat. #78833, Pierce, Rockford, Ill., USA). Similarly, isolation of mitochondrial extracts was performed with the mitochondria isolation kit (Cat. #89874, Pierce), lysosomal extracts with the lysosome enrichment kit for tissue and cultured cells (Cat. #89839, Pierce), and peroxisomal extracts with the peroxisome enrichment kit for tissue (Pierce) following the manufacturer's protocols. Desalting prior to further processing was accomplished by the use of Zeba Desalt Spin columns (Cat. #89890, Pierce). The presence of mRNA in the extract was excluded by using the μ-quant spectrophotometer (Bio-Tek instruments). The extracted proteins were labeled with fluorescein isothiocyanate (FITC) using the EZ Label Fluorescein Protein labeling kit (Cat. #53000, Pierce) and the labeled protein was detectable with fluorescence microscopy for 3-5 days after transfection.

Transfer of Lineage Determining Factors: It was previously shown that human ADSCs have the potential to spontaneously differentiate into a cardiac lineage. However, that capacity diminishes with aging, such that with passage 5, cells no longer express cardiac markers. In order to examine a stimulus-dependent differentiation, only passage 5 and 6 cells were used for protein transfection. Expression of cardiac-specific markers was determined in ADSCs by immunofluorescence and RT-PCR after transfection with various cardiomyocyte and whole-heart-derived proteins.

Cells were plated at a density of 1-2×10⁵ cells per well in a 6-well plate and cultured overnight before transfer (a.k.a. transfection) under normal culture conditions. In order to optimize the transfection conditions, testing was undertaken of amounts equivalent to 5 or 10 μl of protein transfection reagent (Pro-Ject Protein Transfection Kit, Cat. #89850, Pierce) in combination with amounts of extract characterized by 0.1, 1, 2, 3.5, 8, 16 or 20 μg of protein.

Specifically, the protein transfection kit utilized includes a cationic lipid supplied as a dry film. The lipid reagent supplied is dissolved by addition of 250 μl chloroform or methanol and a desired amount, i.e. 5 or 10 μl, of the dissolved lipid in organic solution is placed in each tube and the solvent is evaporated leaving a dry film on a tube wall. The aqueous extract in a volume of 50 μl per 5 μl lipid or 100 μl per 10 μl lipid is added to the dry film and vortexed. The addition of the aqueous solution to the dried lipid results in formation of liposomes that include portions of the aqueous extract sequestered within the interior of the liposomes as well as proteins trapped or adhered to the liposome exterior on the basis of polar or charge interactions.

Data are presented for quantities of 1-2×10⁵ cells transfected with extracts having 3.5 μg of protein and 10 μl of transfection reagent and incubated for 4 hr in serum-free medium. After incubation, the medium was exchanged with 20% FBS-containing medium. Transfection efficiency and viability were subsequently determined by FACS, as well as collection of successfully transfected cells.

Labeling of protein in the extracts made it possible to track them in the transfected cell. Protein extracts from either freshly isolated cardiomyocytes or whole hearts from neonatal rats were labeled with FITC as described above. Using fluorescence microscopy, the labeled particles could be detected after transfection indicating effective protein transfer. The origin of the protein made no difference in the transfection efficiency or the location of proteins. Quantification of transfection efficiency and viability was determined by flow cytometry and FACS.

Of the various cell numbers and densities used, 0.2×10⁶ cells per 6-well at a confluence of 70-80% was the optimal cellular transfection condition. The volume of transfection reagent had a small influence on the efficiency. Volumes of 5 μl and 10 μl of reagent per well (6 well) resulted in similar outcomes of 80-90% transfection. The viability, in contrast, decreased from approximately 90% to 60% using 10 μl of transfection reagent versus 5 μl. However, this was primarily due to protein toxicity not toxicity from the transfection reagent. A protein concentration of 0.1 μg resulted in a FITC labeling of approximately 50%, 1 and 2 μg of about 70% and 3.5 μg of 90%. Although a concentration of 8-20 μg of protein showed a transfection efficiency of 99%, transfection of cells with more than 8 μg of protein per 6-well had a cytotoxic effect on the cells and resulted in poor cell viability (0.8±0.32%). It was noted that FITC labeled proteins from nuclear extracts were shown to proceed not only into the cytoplasm but also into the nuclei.

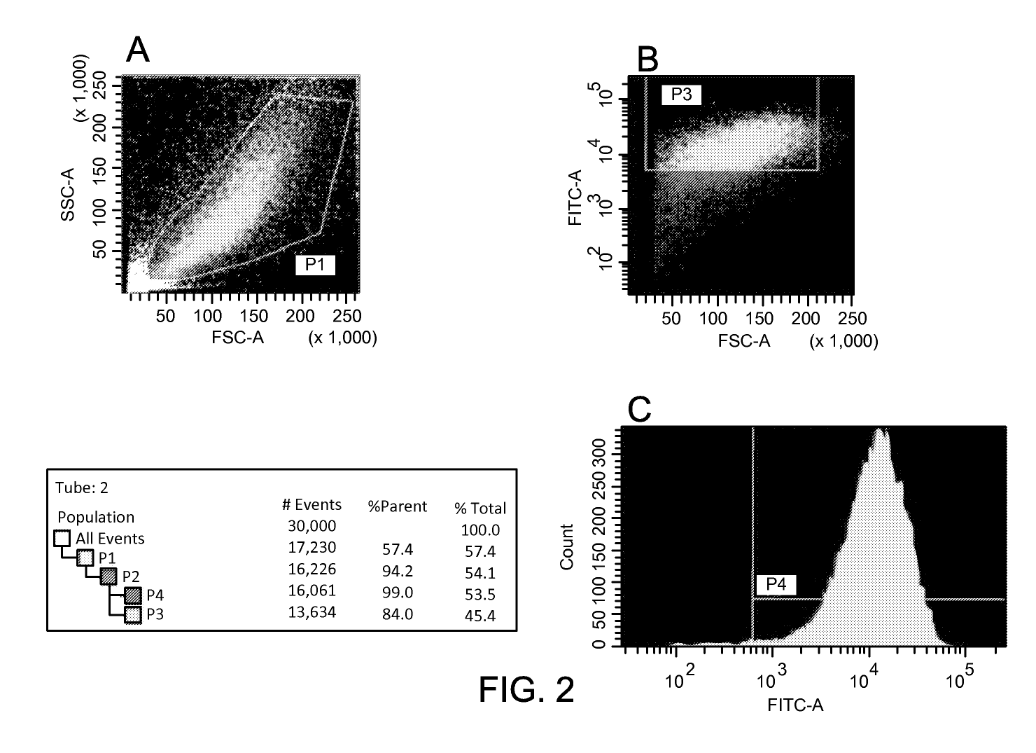

High transfection efficiency (90.14±9.19%) with an almost-unaffected viability (96.9±0.32%) was achieved by using 3.5 μg of protein per 6-well. As depicted in FIG. 2, (A) Cells maintained normal cell shape but (B, C) expressed a bright FITC fluorescence—84% of cells (P3) were significantly brighter than control ADSCs. Control ADSCs which were untreated showed a viability of 98.2±0.43% for ADSCs exposed to the transfection reagent but not to proteins. Due to the high yield of transfected cells, only 40-60% of the transfected cells with the highest fluorescence were used for further culture after separation by FACS. Passage 6 to 12 control ADSCs and ADSCs exposed only to the protein-transfection reagent but no protein showed no evidence of cardiomyogenic, smooth-muscle or endothelial lineage differentiation by PCR or immunofluorescence.

Changes in cell morphology and size could be observed 7 days after transfection. In comparison to control ADSCs, many transfected cells adopted an elongated morphology or developed a thicker cell body shape. Alterations in cell morphology post-transfection were consistent with phenotypic changes observed with subsequent immunofluorescent and PCR studies.

FACS: FACS was initially performed 14 hr after protein transfection. After detaching the cells with 0.25% trypsin, they were washed twice in PBS and the pellet was resuspended in 0.5 ml serum-free medium. The sorting was performed with the BD FACS Aria cell sorter. Cells were sorted by the FITC fluorescence of the transfected protein. The highest fluorescent one third was collected in 20% FBS containing medium and expanded in vitro under standard culture conditions.

For FACS analysis of cell surface phenotype, analysis was performed 14 days (passage 7) after transfection of cells with whole-heart nuclear and cytoplasmic protein and smooth muscle cytoplasmic protein. Antibodies used were anti-troponin I (Abcam) and anti-α-smooth muscle actin-PE conjugated (R&D Systems).

The marker troponin I was shown in ADSCs 14 days after transfection with whole-heart extracts. Significant gain of the mean of the fluorescence intensity (GMnX) was found in cells induced with whole-heart nuclear extracts (12.98±1.76) compared to the control ADSCs (6.73±0.19) (p=0.37). Whole-heart cytoplasmic extract had a higher, but not significant, GMnX-gain (9.05±3.45). This data is consistent with immunofluorescence results showing an especially high inductive potency with nuclear extracts. Isotype matched control antibody showed no staining.

Transfection with proteins extracted with or without proteinase inhibitor (PI) showed no difference in the expression of cardiomyocyte proteins. Independent of the source of the cardiomyocyte or whole-heart protein (nuclear or cytoplasmic), equal results were found in the expression of Nkx2.5 and troponin I.

Smooth-muscle actin was demonstrated in ADSCs 14 days after transfection with whole-heart extracts. Significant gain of the mean of the fluorescence intensity (GMnX) was found in cells induced with whole-heart nuclear and cytoplasmic extracts in keeping with immunofluorescent results indicating a greater inductive potency of these proteins. Smooth-muscle actin expression was demonstrated by a fluorescence intensity gain in cells transfected with whole-heart nuclear extracts (83.42±3.73) (p=0.25) and in cells transfected with whole-heart cytoplasmic extract (80.38±6.52) (p=0.02) compared to the control ADSCs (59.98±7.81).

Immunofluorescence Analysis: Staining was performed after a time period of 1, 2, 3 and 6 weeks corresponding to passages 6, 7, 8 and 12 respectively. All incubation steps were carried out at room temperature unless otherwise stated. Cells used for immunofluorescence were cultured on 12-mm-diameter circular cover slips. For fixation, cell culture medium was aspirated, cover slips were removed and washed with PBS twice before incubating in 4° C. PFA (4%) for 10 min. Following three washes in PBS, non-specific binding sites were blocked by adding blocking solution for 30 min. Staining with the primary antibody was carried out in an incubation chamber to maintain a temperature of 37° C. Primary antibodies used were anti-α-sarcomeric actin (Abcam), anti-cardiac troponin I (Abcam), antitroponin T (Lab Vision, Fremont, Calif., USA), antimyosin light chain (Abcam), anti-Nkx2.5 (R&D Systems), anti-MyoD (Abcam), anti-α-smooth muscle actin (Abcam), anti-smooth muscle myosin heavy chain (Abcam) and anti-VE cadherin (Abcam). After 90 min of incubation, cells were washed three times with PBS. Assuring that the procedure was kept in darkness, cells were incubated with the secondary antibody (FITC- or PE-conjugated) at a dilution of 1:100 or 1:200 for 45 min at 37° C. Two more washing steps were followed by 20 min incubation with Hoechst 33342 dye as a nuclear stain. Finally, cells were washed and coverslips were prepared on slides with mounting medium and dried overnight before sealing. Cell surface markers were analyzed in triplicate and each of the three samples included counting three fields per slide containing about 30-40 cells per field. Isotype control antibodies were used to gate negative populations. The percentage number of specific cell surface markers was obtained by subtracting that of the isotype matched control antibody.

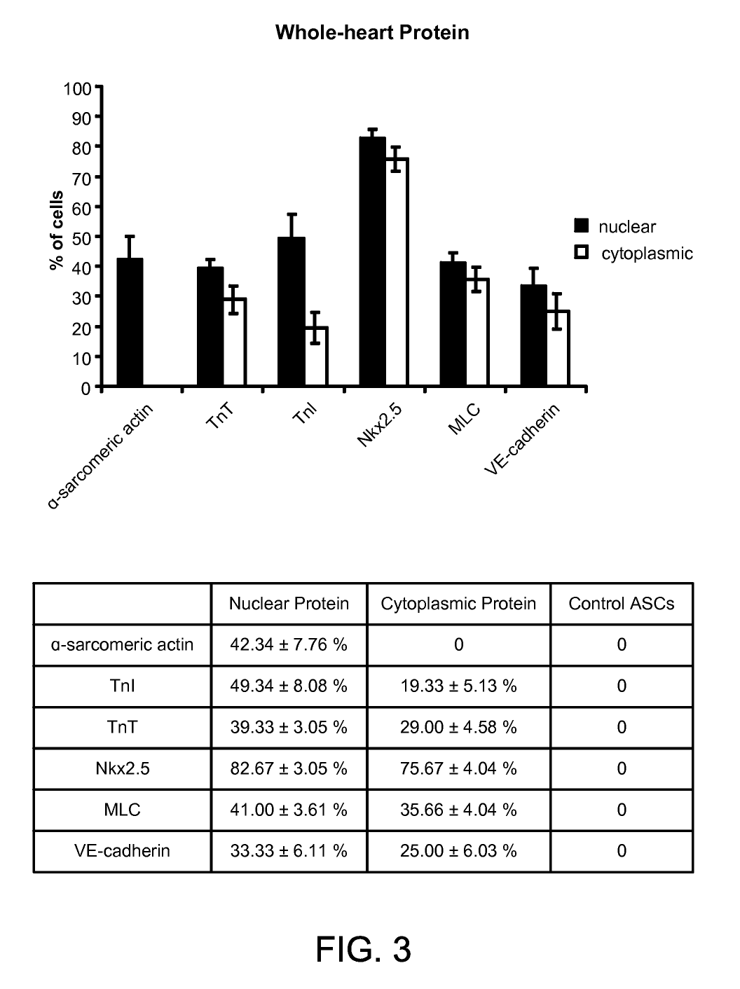

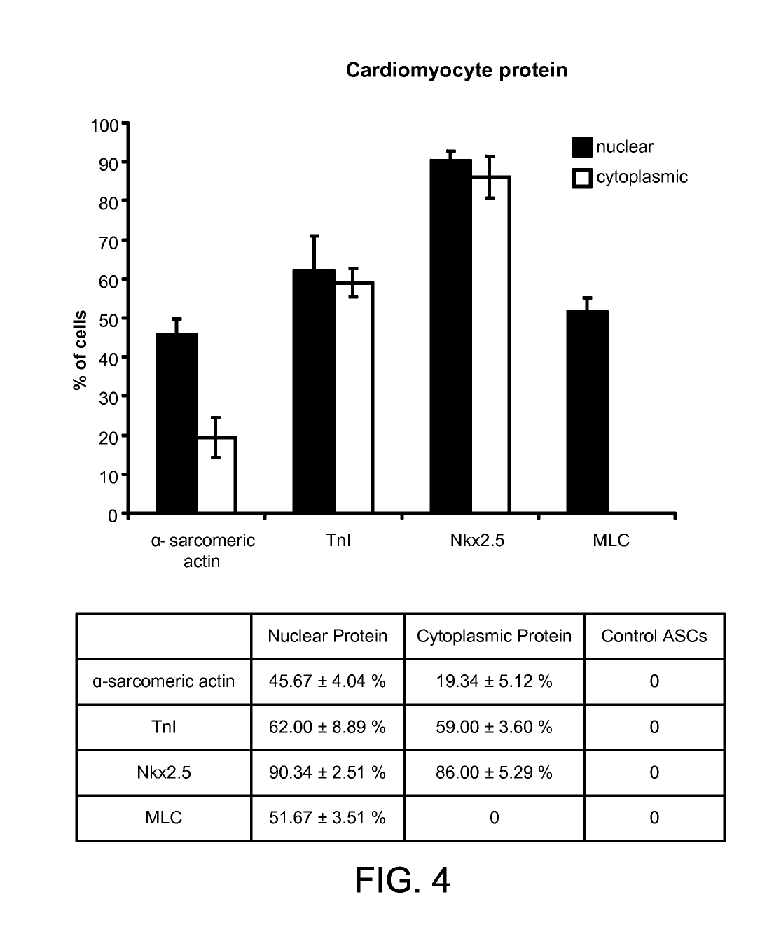

Transfected ADSCs were shown to express the cardiomyogenic markers Nkx2.5, αsarcomeric actin, c-troponin I, c-troponin T, MLC and desmin by 2 weeks after liposome mediated transfection with proteins from neonatal rat cardiomyocytes or whole hearts. As depicted in FIG. 3, Nkx2.5, a transcription factor expressed early in the cardiomyogenic differentiation lineage, showed the highest differentiation rate with 90.34±2.52% positive in cells transfected with cardiomyocyte nuclear protein and 82.67±3.05% positive in cells transfected with whole-heart nuclear protein. In comparison, control ADSCs transfected only with liposomal transfection reagent and not with protein showed 0% positive cells. Comparison of cells induced with proteins derived from different cell elements (cytoplasmic, nuclear, mitochondrial, lysosomal and peroxisomal extracts) revealed that the nuclear and cytoplasmic extracts induce a higher differentiation rate than extracts from the mitochondria, lysosomes or peroxisomes. This observation was made independent of the transfected protein origin, whether from cardiomyocytes or whole-heart extracts. In the case of whole-heart protein transfection, the percentage of expression of Nkx2.5 found in ADSCs was 75.67±4.04% with transfected cytoplasmic protein extracts, 82.67±3.05% with nuclear extracts, 66.62±8.08% with mitochondrial extracts, 56±8.54% with lysosomal extracts and 56.68±7.64% with peroxisomal extracts (p<0.03). A similar trend was observed with troponin I expression in cells transfected with cardiomyocyte proteins. Induction of troponin I expression was observed at a rate of 62±8.80% in cells transfected with nuclear protein extracts and 59±3.61% with cytoplasmic extracts, while mitochondrial (33.35±7.64%) and lysosomal (0%) extracts achieved a significantly lower induction of expression (p<0.001). Though cytoplasmic extracts were shown to have a high inductive capacity, nuclear extracts, independent of origin, proved more potent. The expression of α-sarcomeric actin in cells transfected with whole heart proteins was higher after induction with nuclear extracts (42.34±7.76%) than with cytoplasmic extracts (0%) (p<0.01). Similar findings apply to the expression of troponin I after transfection with whole-heart nuclear extracts (51.58±6.51%) and whole-heart cytoplasmic extracts (19.34±5.03%) (p<0.01). Whole-heart nuclear protein transfection also induced desmin with an expression rate of 46.84±8.08% but whole-heart cytoplasmic protein only at a rate of 23.28±7.64% (p<0.01). Related results were found in cells induced by cardiomyocyte proteins as depicted in FIG. 4. The expression of α-sarcomeric actin in cells transfected with cardiomyocyte nuclear proteins (46.33±4.04%) was greater than with cytoplasmic nuclear proteins (19.34±5.03%) (p<0.02). Isotype-matched control antibody showed no staining nor did the untreated ADSC control. Likewise, staining for MyoD, a marker for skeletal-muscle derivation, was negative, proving that the myogenic differentiation was of cardiogenic and not of skeletal-muscle lineage. Cardiogenic differentiation was observed in cells transfected with various protein extracts from cardiomyocyte or whole-heart extract.

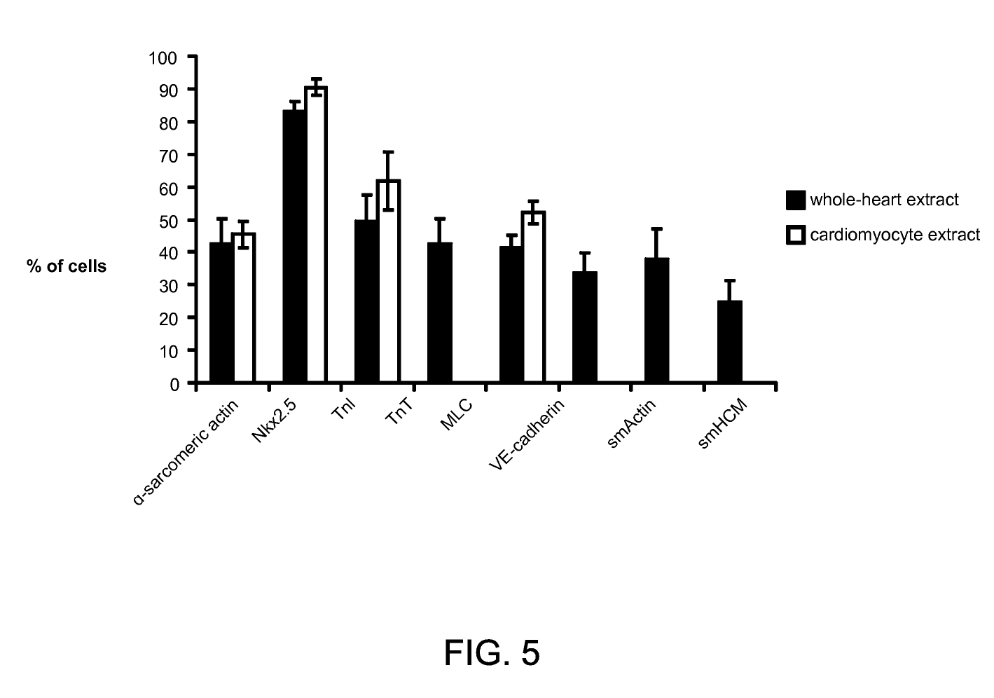

However, cardiomyocyte extracts compared to whole heart extracts had the greater potency in inducing cardiomyogenic differentiation as shown in FIG. 5. In the case of MLC and Nkx2.5 cardiomyocyte nuclear protein induced a higher expression rate than that of whole-heart nuclear protein. Myosin light chain expression was greater with cardiomyocyte nuclear protein transfection (51.67±3.51%) than with whole-heart nuclear proteins (41±3.16%) (p=0.021). Likewise, Nkx2.5 showed the highest differentiation rate with 90.34±2.52% in cells transfected with cardiomyocyte nuclear protein and 82.67±3.05% of cells transfected with whole-heart nuclear protein (p=0.028).

The greater capacity of cardiomyocyte proteins compared to whole-heart proteins was supported by the findings with regard to differential induction capacities of cytoplasmic protein preparations derived from cardiomyocytes alone versus whole-heart cytoplasmic protein preparations. Cardiomyocyte cytoplasmic extracts induced the expression asarcomeric actin in 19.34±5.03% of transfected cells while whole-heart cytoplasmic extracts had no impact (p<0.001). Similarly in the case of desmin, transfection of cardiomyocyte cytoplasmic extracts (41±3.61%) had a greater effect than extracts from whole hearts (23.28±7.64%) (p<0.01). Likewise with troponin I, the induction rate was 59±3.61% in cells transfected with cardiomyocyte cytoplasmic extract and 19.34±5.03% in cells transfected with whole-heart extract (p<0.01).

Troponin T was identified in cells transfected with whole-heart proteins with a high percentage of expression after nuclear extract transfection (39.33±3.06%) versus a somewhat diminished rate of induction after cytoplasmic extract transfection (29±4.58%). Cells transfected with a mix of all protein extracts (independent whether whole-heart or cardiomyocyte) showed expression patterns similar to that of nuclear and cytoplasmic extracts albeit with slightly lower percentages. In the case of troponin T, whole-heart nuclear proteins induced that marker in 39.33±3.06% while the mix showed a percentage of 35.68±4.04%.

RT-PCR Analysis: Reverse transcription-polymerase chain reaction (RTPCR) was performed after 1, 2, 3 and 6 weeks from protein-transfected cells as well as from control ADSCs using the RNeasy mini kit (Qiagen, Valencia, Calif., USA). cDNA was synthesized from 1 mg total RNA using SuperScript II reverse transcriptase (Life Technologies Inc. Gaithersburg, Md., USA) following the manufacturer's instructions. cDNA samples were subjected to PCR amplification using AccuPrime Super Mix I (Invitrogen, Carlsbad, Calif., USA) with primers selective for human cardiac genes including Nkx2.5, troponin I, troponin T, mlc2v (myosin light chain), and GAPDH.

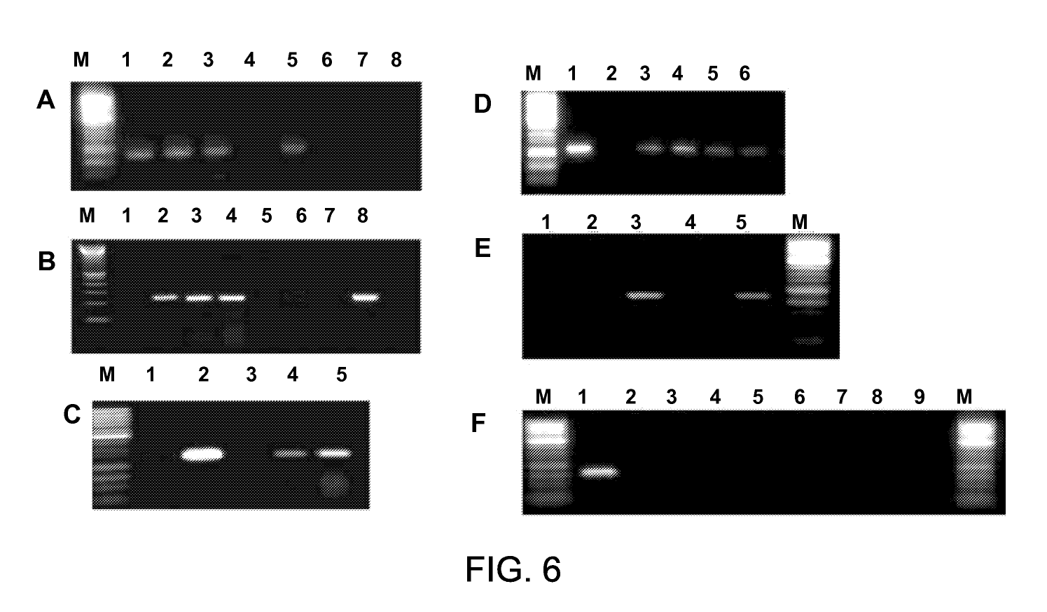

Similar results to that determined by immunofluorescence were obtained in the genetic analysis. Cardiac markers were expressed 7 to 14 days after transfection. Nkx2.5 mRNA expression was detected 7 days after transfection with cardiomyocyte and whole-heart extracts. FIG. 6A shows Nkx2.5 mRNA expression by RT-PCR after transfection of ADSC with Cardiomyocyte proteins (CM): (1)—after 1 week, (2)—after 2 weeks, (3)—after 3 weeks, (4)—after 6 weeks, (5)—heart (positive control), ADSCs exposed to Reagent only: (6)—after 1 week, (7)—after 2 weeks, (8)—after 3 weeks. FIG. 6B shows Troponin I mRNA expression by RT-PCR: (1)—reagent only (2 weeks), (2)—CM at 2 weeks, (3)—whole heart at 2 weeks, (4)—CM at 3 weeks, (5)—whole heart at 3 weeks, (6)—CM at 6 weeks, (7)—whole heart at 6 weeks, (8)—heart (positive control). FIG. 6C shows Troponin T mRNA expression by RT-PCT: (1)—reagent only, (2)—heart, (3)—h lipo p6, (4)—whole heart at 1 week, (5)—whole heart at 2 weeks, (6)—CM at 1 week, (7)—CM at 2 weeks. FIG. 6D shows mlc2v mRNA expression by RT-PCR: (1)—heart (positive control), (2)—control ADSC (passage 6), (3)—whole heart after 1 week; (4)—CM after 1 week; (5)—whole heart after 2 weeks; (6)—CM after 2 weeks. FIG. 6E shows Nkx2.5 mRNA expression by RT-PCR after transfection of ADSC with whole heart proteins: (1)—after 6 weeks, (2)—after 1 week, (3)—after 2 weeks, (4)—after 3 weeks, (5)—heart (positive control). FIG. 6F shows cardiac differentiation manifest by mRNA expression by RT-PCR after 6 weeks: (1)—heart (positive control), (2)—Nkx2.5 CM, (3)—Nkx2.5 whole-heart, (4)—TnI CM, (5)—TnI whole-heart, (6)—TnT CM, (7)—TnT whole-heart, (8)—mlc2v CM, (9)—mlc2v whole-heart.

Consistent with the previous results, troponin I was also expressed independent from the origin of protein after 7 days. Expression of myosin light chain could be observed in cells transfected with whole heart protein and cardiomyocyte nuclear protein, but not through other cardiomyocyte extracts. Likewise, troponin T was detected in cells transfected with whole-heart proteins as already shown by immunofluorescence.

Persistence of Phenotypic Changes: The phenotypic differentiation achieved by liposome transfection eventually waned. In order to evaluate the time course, the expression of specific markers was tracked over a time period of 6 weeks on days 7, 14, 21 and 42. Induction of cardiac lineage-specific markers troponin I, troponin T, Nkx2.5 and myosin light chain (MLC) could be found after 1 week independently of the source of protein. Whole-heart nuclear protein-transfected cells expressed the highest rate of cardiac markers at 2 weeks after transfection. At time-points later than that, there was no remaining evidence of a cardiomyogenic lineage differentiation. In contrast, cardiomyocyte nuclear proteins had a longer impact on phenotypic induction. Troponin I, Nkx2.5 and MLC could be found at time-points up to 3 weeks. However, after 6 weeks, no sign of differentiation could be detected in either group. These data indicate that predifferentiation through transfection with nuclear proteins must be followed by implantation while the desired differentiation phenotype is still being expressed.

Endothelial and smooth muscle lineage differentiation: In addition to the expression of cardiac proteins, endothelial cell and smooth muscle cell markers were found in cells transfected with whole heart proteins. VE-cadherin, a cardiac endothelial marker, was expressed in 33.33±6.1% of cells transfected with nuclear protein and in 25±5.0% of cells transfected with cytoplasmic protein. Mitochondrial, lysosomal and peroxisomal proteins were also shown to induce endothelial-like differentiation but to a less degree. Similar to the cardiomyogenic induction, markers could be shown after 7 days and but were not persistent for more than 3 weeks.

After transfection with extracts from whole hearts, smooth-muscle actin was found in 37.70±9.29% of cells induced with nuclear and 35.33±4.51% of cells induced with cytoplasmic extracts. As with results observed with cardiogenic induction, nuclear and cytoplasmic proteins showed a greater effect than lysosomal, peroxisomal and mitochondrial proteins (p<0.023). Similar findings were obtained for expression of smooth-muscle heavy chain. Nuclear extracts induced 34.53±6.1% and cytoplasmic extracts 24.67±6.7% of cells two weeks after transfection, while the other protein extracts induced expression in less than 17% of the cells (p<0.028).

### Example 3

### Lineage Pre-Determination of ADSC into an Endothelial Cell Phenotype

Lineage predetermination by transfection of ADSC with endothelial cell extracts was also successful. ADSCs transfected with cytoplasmic extracts of endothelial cells were observed to express the endothelial markers CD31 and VE-cadherin. Alterations in cell morphology post-transfection were consistent with phenotypic changes observed with subsequent immunofluorescent and PCR studies. Seven days after transfection with endothelial cytoplasmic protein, VE-cadherin was expressed in 38.33±6.51%, CD31 in 50.67±5.03%, and von Willebrand Factor (vWF) in 56±5.29% of the cells but not in control ADSCs transfected only with the liposomal transfection reagent. Expression of these markers diminished after 2 weeks of culture, most markedly in the case of CD31 (0%) and VE-cadherin (16.67±4.16%). vWF was the only marker in this sub-series that persisted at 2 weeks post-transfection, expressed in cells for 14 days maintaining a similar percentage of expression (53.34±6.11%).

### Example 4

### Lineage Pre-Determination of ADSC into a Smooth-Muscle Cell Phenotype

Transfection of ADSC with smooth-muscle cell cytoplasmic protein stimulated the expression of lineage-specific markers smooth-muscle actin and smooth muscle heavy chain myosin. Desmin, a marker of cardiac and smooth-muscle differentiation, was also observed. After 7 days, smooth-muscle actin was found in 48%±6.0% of cells after transfection, but was not detectable after 14 days. Smooth-muscle heavy chain was present in 51.33±7.57% of cells 7 days after transfection with smooth-muscle cytoplasmic extract. The percentage of expression was stable for 7 more days. 48±5.29% of cells expressed smooth-muscle heavy chain after 14 days. Likewise, desmin was expressed in 47.67±6.67% of cells after 7 days and diminished to 34.67±4.16% cells after 14 days. By FACS analysis, smooth-muscle actin expression was observed in cells 14 days after transfection with smooth-muscle cell cytoplasmic extract. This expression was demonstrated by a gain of fluorescence intensity in transfected cells of 17.28±0.97 compared to non-transfected control ADSCs with 9.92±0.2 (p<0.01).

### Example 5

### Isolation of Fresh Reparative Cells from Adipose Tissue

When the connective tissue of adipose tissue is digested, such as with collagenase, the lipid containing adipocytes can be separated from the other cell types. In 1964, Rodbell reported the use of collagenase to dissociate adipose tissue into a cellular suspension that could then be fractionated by centrifugation into an upper, lipid-filled adipocyte fraction, and a cell pellet comprised of non lipid-filled cells. The pelleted non-adipocyte fraction of cells isolated from adipose tissue by enzyme digestion has been termed the “stromal vascular cell” or “SVF” population. (Rodbell M. “Metabolism of isolated fat cells: Effects of hormones on glucose metabolism and lipolysis” J. Biol. Chem. 239 (1964) 375-380). Typically adipocytes are separated from the SVF by centrifugation wherein the adipocytes float and the cells of the SVF pellet. The SVF is subject to further processing and selection, including plastic adherence. Cells from the stromal vascular fraction that have been subject to plastic adherence are typically referred to as cultured stromal vascular cells or “adipose tissue-derived stromal cells” (ADSC). Not withstanding other definitions that may exist in the art, as used herein, the term “stromal vascular fraction cells” refers to all of the constituent cells of adipose tissue after enzyme digestion and removal of adipocytes and are not limited to plastic adherent cells.

Researchers have studied the makeup of the stromal vascular fraction of adipose tissue across a range of disciplines. Typically, the stromal vascular fraction cells that are adherent have comprised the population that has been studied in culture. In addition to fibroblasts, the stromal vascular fraction of adipose tissue has been shown to contain, among other cell types, microvessel endothelial cells, vascular progenitor cells, adipocyte progenitor cells (preadipocytes), and multipotent progenitor cells. Subsequent to Rodbell's original isolation, others, using in vitro and in vivo models, identified cells within the SVF that could differentiate into adipocytes. These cells were termed preadipocytes and were identified as plastic adherent cells within the SVF.

Plastic adherent cells from the SVF have been shown capable of differentiation into multiple lineages including adipocytes, preadipocytes, osteoblasts, and chondrocytes, The ability of plastic adherent SVF cells to differentiate into multiple lineages fits the criteria of multipotent mesenchymal stem cells. (See review by Zuk et al “Human Adipose Tissue is a Source of Multipotent Stem Cells” Mol. Biol. Cell 13 (2002) 4279-95).

In one embodiment of the invention, a population of cells for cell transplantation is prepared without the centrifugation step of the prior isolation methods. A sample of donor adipose tissue is dissociated by enzymatic digestion into individual cells and small clusters of cells until the dissociated cells and clusters of cells are reduced in diameter to about 1000 microns or less. The individual cells and small clusters of cells are phase separated into an aqueous cellular layer and a lipid layer without centrifugation, and cells for cell transplantation are collected from the aqueous cellular layer. In one embodiment of the invention the phase separation is undertaken by introducing the dissociated cells, including adipocytes, into a lipid separating unit in an aqueous medium. The lipid and lipid containing adipocytes float upward thus forming a top lipid layer in the lipid separating unit while the non-lipid containing or non-adipocyte cells float downward under the influence of normal gravity and are withdrawn from under the top lipid layer. In accordance with this method, non-adipocytes can be separated from lipid containing cells without centrifugation.

In one particular embodiment of the invention, adipose tissue is introduced into a digestion chamber that includes a digestion fluid and an internal digestion mesh and the tissues and digested cells are recirculated across the digestion mesh until the tissue is separated into a digestion mixture that includes individual cells and small cell clusters, followed by phase separating the digestion mixture through an aqueous medium disposed in a lipid separation unit. Isolation of desired cell populations is preferably accomplished in a unitary device without a need for centrifugation. In further embodiments, the digestion mixture is filtered over at least one dispersing filter prior to phase separating. In certain embodiments the digestion mixture is finally conveyed through a dispersing head that is disposed within and forms an entry port to the lipid separating unit. The dispersing head further divides clumps of cells within the digestion mixture as the digestion mixture enters the lipid separation unit. The method is particularly suitable isolation of cells from adipose-containing tissues of human, equine, canine, feline, simian, caprine, and ovine origin.

In one example, a reparative cell population is isolated as follows. Lipoaspirate is collected under informed consent in the operating room directly into a unitary purification apparatus by standard suction assisted lipoplasty with tumescent. The digestion chamber of the apparatus includes a predigestion chamber and an inner postdigestion chamber separated by a nylon mesh having a pore size of approximately 1 mm. The tumescent is drained and a volume of approximately 100 ml of drained lipoaspirate is washed by draining the predigestion chamber and refilling with a solution of lactated Ringer's solution, which was prewarmed to 37° C. and contains a proteolytic enzyme combination comprised of collagenase IV (60,000 U) and dispase (120 U). An additional 150 ml of lactated Ringer's is added to the lipid separating unit. The digestion recirculation loop is implemented by a pump actuated flow path from the predigestion chamber into the postdigestion chamber and including passage across a heat exchanger that maintains the digestion mixture at approximately 37° C. Recirculation is continued for approximately 30 to about 60 minutes or until greater than 90% of the cellular volume of the predigestion chamber is able to pass the 1 mm mesh into the post digestion chamber. The design of the pre and post digestion chambers, separated by the nylon mesh across which the recirculation flow path passes repeatedly, provides trapping of connective and other debris tissue on the digestion mesh. After digestion is sufficiently complete, the digestion mixture is pumped tangentially over a nylon dispersing filter having a pore size of 250 μm. The filtered digestion mixture is then pumped into a columnar lipid separating chamber that is integral to the apparatus. As previously mentioned, the lipid separating chamber is prefilled with a volume of 150 ml lactated Ringer's solution prior to introduction of the digestion mixture such that when the filtered digestion mixture enters the chamber, any clusters of cells, including lipids or adipocytes, are subject to fluid shear as the lipid moieties float upward in the aqueous solution. In one embodiment, the filtered digestion mixture enters the lipid separating chamber through a dispersing head having a plurality of downwardly directed pores with a pore size of 500 μm and disposed proximally to a bottom inner surface of the lipid separating unit. The design of the dispersing head is adapted for forcibly flowing the cell mixture against an inner surface within the lipid separating unit and thereby further disrupting cell clusters within the cell mixture prior to fluid phase separation. Fluid phase separation is allowed to proceed at room temperature for about 5 to about 30 minutes prior to collection of the fresh stromal vascular fraction from the bottom of the lipid separating chamber.

The freshly isolated SVF cells may be further subject to plastic adherence to isolate the adherent cells classically defined as mesenchymal stromal cells. The adherent cells can be liposome transfected with differentiated cell extracts after overnight culture or after several passages. Alternatively, in one embodiment of the invention, freshly isolated SVF cells are immediately liposome transfected with differentiated cell extracts. In one embodiment of the invention, the transfected cells are immediately reimplanted into the autologous donor. Such embodiments provide a lineage pre-determined reparative cell preparation for cell therapy, wherein the cell preparation comprises a heterogeneous mixture of tissue engrafting cells and supportive cells that is derived without prior expansion in cell culture. In one embodiment of the invention, the isolated SVF cells are divided into aliquots and each aliquot is transfected with cell extract of different lineages. For example, in the treatment of myocardial infarction, one aliquot is transfected with a cardiomyocyte extract while other extracts are transfected with smooth muscle, and/or endothelial extracts. After transfection, the aliquots are combined for site specific injection into an area of infarcted myocardium. The various lineage pre-determined populations are expected to work in concert to restore the damaged myocardium to normal function.

In other embodiments, the transfected SVF cells are cultured to separate into plastic adherent and non-adherent populations and further cultured for expansion if desired. Once derived, cell preparations of the present invention can optionally undergo further treatment prior to use for cell therapy. For instance, in one example, leukocytes within the cell preparation may be removed. In further examples, cell preparations of the present invention are seeded (i.e., applied) onto a biocompatible matrix and are then suitable for implantation at the point-of-care. Such biocompatible matrices can include without limitation scaffolds, grafts, sponges, and other well known materials that may be surgically implanted into the subject.

Methods and compositions for the generation of matrices that are seeded with the largest obtainable numbers of lineage committed cells have not been heretofore available and there continues to be an unmet need for implantable cell seeded matrices where the reparative cell populations or isolated stem cell populations are treated with lineage determining factors prior to implantation. In this way, the cells receive a jump start toward differentiation into the desired phenotype. In some embodiments, the cells are seeded onto the scaffold material after treatment with lineage determining factors. In other embodiments, the reparative cell populations or isolated stem cell populations are seeded onto the matrix and then treated with the lineage determining factors.

In either event, the lineage determining factor treated cells, which are seeded onto matrices may be immediately implanted or, alternatively, cultured prior to implantation. The culture period may be from 1 day to several weeks depending on the indication and need for cell expansion. In certain embodiments, the lineage determining factor treated cells are incubated with inductive media before, during or after being applied to the scaffold. For example, the inductive media may be adapted for generation of one or more of adipocytes, chondrocytes, endothelial cells, hepatocytes, myoblasts, Schwann cells, pancreatic islet cells, hematopoietic cells, myeloblasts, neurons, and osteoblasts.

The present invention provides methods and materials for the focal application of precommitted ADSC and reparative cell populations, for example for repair of damaged neurons, muscle, tendons, joints and bone structures, repair of parenchymal organs such as liver, kidney, heart, or brain, and for repair of skin tissues including in the treatment of burns, hernias, and non-healing wounds.

All publications, patents and patent applications cited herein are hereby incorporated by reference as if set forth in their entirety herein. While this invention has been described with reference to illustrative embodiments, this description is not intended to be construed in a limiting sense. Various modifications and combinations of illustrative embodiments, as well as other embodiments of the invention, will be apparent to persons skilled in the art upon reference to the description. It is therefore intended that the appended claims encompass such modifications and enhancements.

We claim:

1. A method of generating an isolated population of lineage pre-determined cells, comprising:
isolating a recipient cell population that is enriched in cells that are characterized by phenotypic plasticity;
transfecting the recipient cell population with a liposome composition comprising a lineage predetermining protein extract obtained from differentiated donor cells;
obtaining a population of transfected recipient cells that express at least one lineage predetermined marker derived from the differentiated donor cells.

2. The method of claim 1, wherein the recipient cell population is enriched in bone marrow or adipose derived mesenchymal stem cells.

3. The method of claim 1, wherein the lineage predetermining cell extract is a cytoplasmic extract obtained from the differentiated donor cells.

4. The method of claim 1, wherein the lineage predetermining extract is a nuclear extract obtained from the differentiated donor cells.

5. The method of claim 1, wherein the lineage predetermining extract is obtained from xenogeneic differentiated donor cells.

6. The method of claim 1, wherein the lineage predetermining extract is obtained from allogeneic differentiated donor cells.

7. The method of claim 1, wherein the recipient cell population is a freshly prepared adipose stromal vascular cell fraction.

8. The method of claim 1, wherein the lineage predetermining protein extract is obtained from a population of differentiated donor cells selected from the group consisting of: adipocytes, chondrocytes, endothelial cells, hepatocytes, cardiomyocytes, smooth muscle cells, skeletal muscle cells, cardiac pacemaker cells, Schwann cells, pancreatic islet cells, hematopoietic cells, myeloblasts, neurons, and osteoblasts.

9. The method of claim 1, wherein the liposome composition is combined with a biocompatible or biodegradable scaffold.

10. The method of claim 1, further comprising culturing the population of transfected recipient cells in a differentiation induction media.

11. A method of treating a diseased tissue in a patient with a lineage pre-determined cell transplant comprising:
removing a sample of adipose or bone marrow tissue from the patient and isolating from the tissue a recipient cell population that is enriched in cells characterized by phenotypic plasticity;
treating the recipient cell population with a liposome composition comprising a lineage predetermining protein cell extract, whereby a significant proportion of the cell population is induced to express at least one lineage predetermined marker as a consequence of the treatment;
implanting the treated recipient cell population into the diseased tissue of the patient.

12. The method of claim 11, wherein the diseased tissue is a tissue damaged by myocardial infarction.

13. The method of claim 11, wherein the lineage predetermining protein cell extract is obtained from a population of differentiated donor cells selected from the group consisting of: adipocytes, chondrocytes, endothelial cells, hepatocytes, cardiomyocytes, smooth muscle cells, skeletal muscle cells, cardiac pacemaker cells, Schwann cells, pancreatic islet cells, hematopoietic cells, myeloblasts, neurons, and osteoblasts.

14. The method of claim 11, wherein the lineage predetermining cell extract is obtained from xenogeneic differentiated donor cells.

15. The method of claim 11, wherein the lineage predetermining cell extract is obtained from allogeneic differentiated donor cells.

16. The method of claim 11, wherein the recipient cell population is a freshly prepared adipose stromal vascular cell fraction.

17. The method of claim 11, wherein the liposomal composition is combined with a biocompatible or biodegradable scaffold and the recipient cell population is treated with the liposome composition by contact with the scaffold.

18. A method of preparing a lineage predetermining composition comprising,
preparing an aqueous protein extract from a population of differentiated mammalian cells, or cellular fractions thereof, wherein the differentiated mammalian cells are enriched in one or more of adipocytes, chondrocytes, endothelial cells, hepatocytes, cardiomyocytes, smooth muscle cells, skeletal muscle cells, cardiac pacemaker cells, Schwann cells, pancreatic islet cells, hematopoietic cells, myeloblasts, neurons, and osteoblasts;
adding the aqueous protein extract to a dry lipid film and mixing until a protein extract liposomal solution is formed; and
characterizing and standardizing liposomal solution.

19. The method of claim 18, wherein the cellular fraction is a nuclear fraction.

20. The method of claim 18, wherein the protein extract liposomal solution is incorporated into a biomaterial that is combinable with cells and adapted for implantation into a patient.

21. A method of providing a cardiac cell transplant to a patient in need thereof comprising:
removing a sample of adipose tissue from the patient and isolating from the tissue a stromal vascular fraction of cells;
transfecting cells of the stromal vascular fraction with a liposome composition comprising a cardiovascular lineage predetermining protein cell extract, whereby a significant proportion of the transfected cells are induced to express at least one cardiovascular cell lineage predetermined marker as a consequence of the transfection; and
implanting the transfected stromal vascular cells into a site of damaged cardiac tissue.

22. The method of claim 21, wherein the cells of the stromal vascular fraction that are transfected are plastic adherent cells.

23. The method of claim 21, wherein the cardiovascular lineage predetermining protein cell extract is a xenogenic cell extract.

24. The method of claim 21, wherein the cardiovascular lineage predetermining protein cell extract is an allogenic cell extract.

25. A biocompatible or biodegradable cell scaffold comprising a liposome composition that includes at least one lineage predetermining protein cell extract.

26. The biocompatible or biodegradable cell scaffold of claim 25, wherein the biocompatible material is selected from the group consisting of: polytetrafluoroethylene, woven polyester, spun silicone, open foam silicone encased in microporous expanded PTFE, stainless steel, polypropylene, polyurethane, polycarbonate, nickel titanium shape memory alloys and cobalt-chromium-nickel alloys, and combinations thereof.

27. The biocompatible or biodegradable cell scaffold of claim 25, wherein the biodegradable material is selected from the group consisting of: silk fibroin-chitosan, acellular dermal matrices, collagen, polyglactin, and hyaluronic acid.
